(19) United States
(12) Patent Application Publication
(10) Pub. No.: US 2017/0035357 A1
(43) Pub. Date: Feb. 9, 2017

(54) CATHETER WITH INDUCTIVE FORCE SENSING ELEMENTS

(71) Applicant: Boston Scientific Scimed Inc., Maple Grove, MN (US)

(72) Inventors: Darrell L. Rankin, Milpitas, CA (US); John C. Potosky, San Jose, CA (US)

(21) Appl. No.: 15/230,314

(22) Filed: Aug. 5, 2016

Related U.S. Application Data
(60) Provisional application No. 62/202,324, filed on Aug. 7, 2015.

(51) Int. Cl. A61B 5/00; A61B 18/14; A61B 5/02

(52) U.S. Cl. CPC A61B 5/6852; A61B 5/02; A61B 18/1492; A61B 5/742; A61B 2018/00988

(57) ABSTRACT

Various embodiments concerns a system for measuring a force within a body comprising a catheter, the catheter comprising at least one sensor and an element located within the catheter, the element displaceable within the catheter relative to the at least one sensor. The system further comprises control circuitry configured to measure, for each of the at least one sensor, a change in a resonance frequency of the sensor based on a change in distance between the sensor and the element, the change in distance responsive to the force. The control circuitry is further configured to calculate at least one parameter of the force based on the change in the resonance frequency, and output an indication of the at least one parameter of the force.

### CROSS-REFERENCE TO RELATED APPLICATION

This application claims priority to Provisional Application No. 62/202,324, filed Aug. 7, 2015, which is herein incorporated by reference in its entirety.

### TECHNICAL FIELD

The present disclosure relates generally to various force sensing catheter features.

### BACKGROUND

In ablation therapy, it may be useful to assess the contact between the ablation element and the tissue targeted for ablation. In interventional cardiac electrophysiology (EP) procedures, for example, the contact can be used to assess the effectiveness of the ablation therapy being delivered. Other catheter-based therapies and diagnostics can be aided by knowing whether a part of the catheter contacts targeted tissue, and to what degree the part of the catheter presses on the targeted tissue. The tissue exerts a force back on the catheter, which can be measured to assess the contact and the degree to which the catheter presses on the targeted tissue.

The present disclosure concerns, among other things, systems for measuring a force with a catheter.

### SUMMARY

The present disclosure relates to devices, systems, and methods for measuring a force experienced by a catheter.

Example 1 is a system for measuring a force within a body, the system including a catheter and control circuitry. The catheter includes at least one sensor and at least one mass of high magnetic permeability material. The catheter is configured such that, responsive to the force, the at least one mass is displaceable within the catheter relative to the at least one sensor. The control circuitry is configured to measure, for each sensor, a change in a resonance frequency of the sensor based on a change in distance between the sensor and the at least one mass, the change in distance responsive to the force; and to calculate at least one parameter of the force based on the change in the resonance frequency.

In Example 2, the system of Example 1, further comprising a display, wherein the at least one parameter comprises a magnitude and a direction of the force and the control circuitry is configured to graphically indicate on the display the magnitude and the direction of the force.

In Example 3, the system of either of Examples 1 or 2, wherein the high magnetic permeability material has a relative permeability greater than 1500.

In Example 4, the system of any of Examples 1-3, wherein the at least one mass of high magnetic permeability material is passive and is not configured to be electrically energized in connection with measuring the force.

In Example 5, the system of any of Examples 1-4, wherein each of the at least one sensor includes an LC circuit, each at least one LC circuit comprising an inductor and a capacitor which are electrically in parallel, wherein the catheter is configured such that, responsive to the force, either of the at least one mass or each inductor of the at least one LC circuit is displaceable within the catheter relative to the other of the at least one mass or each inductor of the at least one LC circuit.

In Example 6, the system Example 5, wherein the control circuitry is configured to measure, for each of the at least one sensor, the change in the resonance frequency of the sensor based on a change in distance between the inductor of the at least one LC circuit and the at least one mass, the change in distance responsive to the force.

In Example 7, the system of either of the Examples 5 or 6, wherein the catheter further comprises a spring element located between each inductor of the at least one LC circuit and the at least one mass.

In Example 8, the system of Example 7, wherein the spring element is configured to permit the change in distance between each inductor of the at least one LC circuit and the at least one mass, and resiliently reverse the change in distance upon removal of the force from the catheter.

In Example 9, the system of any of Examples 5-8, wherein the at least one LC circuit comprises three LC circuits, the three LC circuits circumferentially arrayed within the catheter.

In Example 10, the system of any of Examples 5-9, further comprising an additional mass of high magnetic permeability material, wherein the at least one mass is positioned either of proximal or distal with respect to the at least one LC circuit and the additional mass is positioned the other of proximal or distal with respect to the at least one LC circuit, and wherein the catheter is configured such that the additional mass is not displaceable within the catheter relative to each inductor of the at least one LC circuit.

In Example 11, the system of any of Examples 5-10, further comprising a printed circuit board, where each of the at least one LC circuit is mounted on the printed circuit board.

In Example 12, the system of Example 11, wherein each inductor of the at least one LC circuit comprises a conductor formed into a flat radial spiral.

In Example 13, the system of any of Examples 5-12, wherein, for each of the at least one LC circuit, the control circuitry is configured to deliver a plurality of pulses, wherein each pulse causes the LC circuit to oscillate, and the control circuitry is configured to measure the change in the resonance frequency by analyzing the oscillation in the LC circuit and determining whether the oscillation changes in frequency between pulses of the plurality of pulses.

In Example 14, the system of any of Examples 5-12, wherein for each of the at least one LC circuit, the control circuitry is configured to deliver a continuous waveform, wherein the continuous waveform causes the LC circuit to oscillate; and the control circuitry is configured to measure the change in the resonance frequency by analyzing the oscillation in the LC circuit and determining whether the oscillation changes in frequency between pulses of the plurality of pulses.

In Example 15, the system of any of Examples 5-14, wherein, for each LC circuit of the plurality of LC circuits, the inductance of the LC circuit changes based on the proximity of the at least one mass to the inductor of the LC circuit.

Example 16 is a system for measuring a force within a body, the system including a catheter and control circuitry. The catheter includes at least one LC circuit and at least one mass of high magnetic permeability material. Each LC circuit of the at least one LC circuit includes an inductor and a capacitor electrically in parallel, wherein the catheter is configured such that, responsive to the force, either of the at least one mass or each inductor of the at least one LC circuit is displaceable within the catheter relative to the other of the at least one mass or each inductor of the at least one LC circuit. The control circuitry is configured to measure, for each of the at least one LC circuit, a change in a resonance frequency of the LC circuit based on a change in distance between the inductor and the at least one mass, the change in distance responsive to the force; and to calculate at least one parameter of the force based on the change in the resonance frequency.

In Example 17, the system of Example 16, wherein the at least one parameter comprises a magnitude and a direction of the force.

In Example 18, the system of Example 17, further comprising a display, wherein the control circuitry is configured to graphically indicate on the display the magnitude and the direction of the force.

In Example 19, the system of any of Examples 16-18, wherein the catheter further comprises a spring element located between each inductor of the at least one LC circuit and the at least one mass.

In Example 20, the system of Example 19, wherein the spring element is configured to permit the change in distance between each inductor of the at least one LC circuit and the at least one mass, and to resiliently reverse the change in distance upon removal of the force from the catheter.

In Example 21, the system of either of Examples 19 or 20, wherein the control circuitry is configured to calculate the parameter of the force by using a function which relates the change in resonance frequency to the change in distance.

In Example 22, the system of any of Examples 19-21, wherein the control circuitry is configured to calculate the parameter of the force by using a constant which relates the change in the change in distance to a value of the parameter of the force.

In Example 23, the system of any of Examples 16-22, wherein the at least one LC circuit comprises three LC circuits, the three LC circuits circumferentially arrayed within the catheter.

In Example 24, the system of any of Examples 16-23, wherein the high magnetic permeability material has a relative permeability greater than 1500.

In Example 25, the system of any of Examples 16-24, wherein the at least one mass of high magnetic permeability material is passive and is not configured to be electrically energized in connection with measuring the force.

In Example 26, the system of any of Examples 16-25, further comprising an additional mass of high magnetic permeability material, wherein the at least one mass is positioned either of proximal or distal with respect to the at least one LC circuit and the additional mass is positioned the other of proximal or distal with respect to the at least one LC circuit, and wherein the catheter is configured such that the additional mass is not displaceable within the catheter relative to each inductor of the at least one LC circuit.

In Example 27, the system of any of Examples 16-25, wherein for each of the at least one LC circuit, the control circuitry is configured to deliver a continuous waveform, wherein the continuous waveform causes the LC circuit to oscillate; and the control circuitry is configured to measure the change in the resonance frequency by analyzing the oscillation in the LC circuit and determining whether the oscillation changes in frequency between pulses of the plurality of pulses.

In Example 28, the system of any of Examples 16-27, further comprising a printed circuit board, where each of the at least one LC circuit is mounted on the printed circuit board.

In Example 29, the system of Example 28, wherein each inductor of the at least one LC circuit comprises a conductor formed into a flat radial spiral.

In Example 30, the system of any of Examples 16-29, wherein, for each of the at least one LC circuit, the control circuitry is configured to deliver a plurality of pulses, wherein each pulse causes the LC circuit to oscillate, and the control circuitry is configured to measure the change in the resonance frequency by analyzing the oscillation in the LC circuit and determining whether the oscillation changes in frequency between pulses of the plurality of pulses.

In Example 31, the system of any of Examples 16-30, wherein, for each LC circuit of the plurality of LC circuits, the inductance of the LC circuit changes based on the proximity of the at least one mass to the inductor of the circuit.

Example 32 is a system for measuring a force within a body, the system including a catheter and control circuitry. The catheter includes at least one sensor, at least one mass of high magnetic permeability material, and at least one spring element. The spring element is configured to permit movement within the catheter between the at least one sensor and the at least one mass responsive to the force. The control circuitry is configured to measure, for each sensor, a change in a resonance frequency of the sensor based on a change in distance between the sensor and the at least one mass, the change in distance responsive to the force; and to calculate at least one parameter of the force based on the change in the resonance frequency.

In Example 33, the system of Example 31, wherein the at least one parameter comprises a magnitude and a direction of the force and the control circuitry is configured to graphically indicate on the display the magnitude and the direction of the force.

In Example 34, the system of either Examples 32 or 33, wherein the control circuitry is configured to calculate the at least one parameter of the force based at least in part on a spring constant for the spring element.

Example 35 is a system for measuring a force within a body, the system including a catheter and control circuitry. The catheter includes at least one sensor and an element located within the catheter. The element is displaceable within the catheter relative to the at least one sensor. The control circuitry is configured to measure, for each of the at least one sensor, a change in a resonance frequency of the sensor based on a change in distance between the sensor and the element, the change in distance responsive to the force; calculate at least one parameter of the force based on the change in the resonance frequency; and output an indication of the at least one parameter of the force

While multiple embodiments are disclosed, still other embodiments of the present invention will become apparent to those skilled in the art from the following detailed description, which shows and describes various illustrative embodiments of the present disclosure. Accordingly, the drawings and detailed description are to be regarded as illustrative in nature and not restrictive.

### BRIEF DESCRIPTION OF THE DRAWINGS

FIGS. 1A-C show a system for measuring a force with a catheter in accordance with various embodiments of this disclosure.

FIG. 2 shows a block diagram of circuitry for controlling various functions described herein.

FIG. 3 shows a detailed perspective view of a distal end of a catheter in accordance with various embodiments of this disclosure.

FIG. 4 shows a perspective view of the inside of a catheter in accordance with various embodiments of this disclosure.

FIG. 5 shows a perspective view of a force measurement assembly that can be housed within a catheter in accordance with various embodiments of this disclosure.

FIG. 6 shows a perspective view of a sensor support in accordance with various embodiments of this disclosure.

FIGS. 7A-7C show alternative embodiments of spring element in accordance with various embodiments of this disclosure.

FIG. 8 shows a perspective view of a portion of a force measurement assembly in accordance with various embodiments of this disclosure.

FIG. 9 shows a printed circuit board containing three LC circuits which operate as components of force sensors in accordance with various embodiments of this disclosure.

FIG. 10 is a schematic illustration showing operational aspects of a force measurement assembly in accordance with various embodiments of this disclosure.

FIG. 11 is a schematic circuit diagram for supporting force sensing functionality in accordance with various embodiments of this disclosure.

FIG. 12 is another schematic circuit diagram for supporting force sensing functionality in accordance with various embodiments of this disclosure.

While the scope of the present disclosure is amenable to various modifications and alternative forms, specific embodiments have been shown by way of example in the drawings and are described in detail below. The intention, however, is not to limit the scope of the invention to particular embodiments described and/or shown. On the contrary, the invention is intended to cover all modifications, equivalents, and alternatives falling within the scope of the appended claims.

### DETAILED DESCRIPTION

Various cardiac abnormalities can be attributed to improper electrical activity of cardiac tissue. Such improper electrical activity can include, but is not limited to, generation of electrical signals, conduction of electrical signals, and/or mechanical contraction of the tissue in a manner that does not support efficient and/or effective cardiac function. For example, an area of cardiac tissue may become electrically active prematurely or otherwise out of synchrony during the cardiac cycle, thereby causing the cardiac cells of the area and/or adjacent areas to contract out of rhythm. The result is an abnormal cardiac contraction that is not timed for optimal cardiac output. In some cases, an area of cardiac tissue may provide a faulty electrical pathway (e.g., a short circuit) that causes an arrhythmia, such as atrial fibrillation or supraventricular tachycardia. In some cases, inactivated tissue (e.g., scar tissue) may be preferable to malfunctioning cardiac tissue.

Cardiac ablation is a procedure by which cardiac tissue is treated to inactivate the tissue. The tissue targeted for ablation may be associated with improper electrical activity, as described above. Cardiac ablation can lesion the tissue and prevent the tissue from improperly generating or conducting electrical signals. For example, a line, a circle, or other formation of lesioned cardiac tissue can block the propagation of errant electrical signals. In some cases, cardiac ablation is intended to cause the death of cardiac tissue and to have scar tissue reform over the lesion, where the scar tissue is not associated with the improper electrical activity. Lesioning therapies include electrical ablation, radio frequency ablation, cyroablation, microwave ablation, laser ablation, and surgical ablation, among others. While cardiac ablation therapy is referenced herein as an exemplar, various embodiments of the present disclosure can be directed to ablation of other types of tissue and/or to non-ablation diagnostic and/or catheters that deliver other therapies.

Ideally, an ablation therapy can be delivered in a minimally invasive manner, such as with a catheter introduced to the heart through a vessel, rather than surgically opening the heart for direct access (e.g., as in a maze surgical procedure). For example, a single catheter can be used to perform an electrophysiology study of the inner surfaces of a heart to identify electrical activation patterns. From these patterns, a clinician can identify areas of inappropriate electrical activity and ablate cardiac tissue in a manner to kill or isolate the tissue associated with the inappropriate electrical activation. However, the lack of direct access in a catheter-based procedure may require that the clinician only interact with the cardiac tissue through a single catheter and keep track of all of the information that the catheter collects or is otherwise associated with the procedure. In particular, it can be challenging to determine the location of the therapy element (e.g., the proximity to tissue), the quality of a lesion, and whether the tissue is fully lesioned, under-lesioned (e.g., still capable of generating and/or conducting unwanted electrical signals), or over-lesioned (e.g., burning through or otherwise weakening the cardiac wall). The quality of the lesion can depend on the degree of contact between the ablation element and the targeted tissue. For example, an ablation element that is barely contacting tissue may not be adequately positioned to deliver effective ablation therapy. Conversely, an ablation element that is pressed too hard into tissue may deliver too much ablation energy or cause a perforation of the cardiac wall.

The present disclosure concerns, among other things, methods, devices, and systems for assessing a degree of contact between a part of a catheter (e.g., an ablation element) and tissue. Knowing the degree of contact, such as the magnitude and the direction of a force generated by contact between the catheter and the tissue, can be useful in determining the degree of lesioning of the targeted tissue. Information regarding the degree of lesioning of cardiac tissue can be used to determine whether the tissue should be further lesioned or whether the tissue was successfully ablated, among other things. Additionally or alternatively, an indicator of contact can be useful when navigating the catheter because a user may not feel a force being exerted on the catheter from tissue as the catheter is advanced within a patient, thereby causing vascular or cardiac tissue damage or perforation.

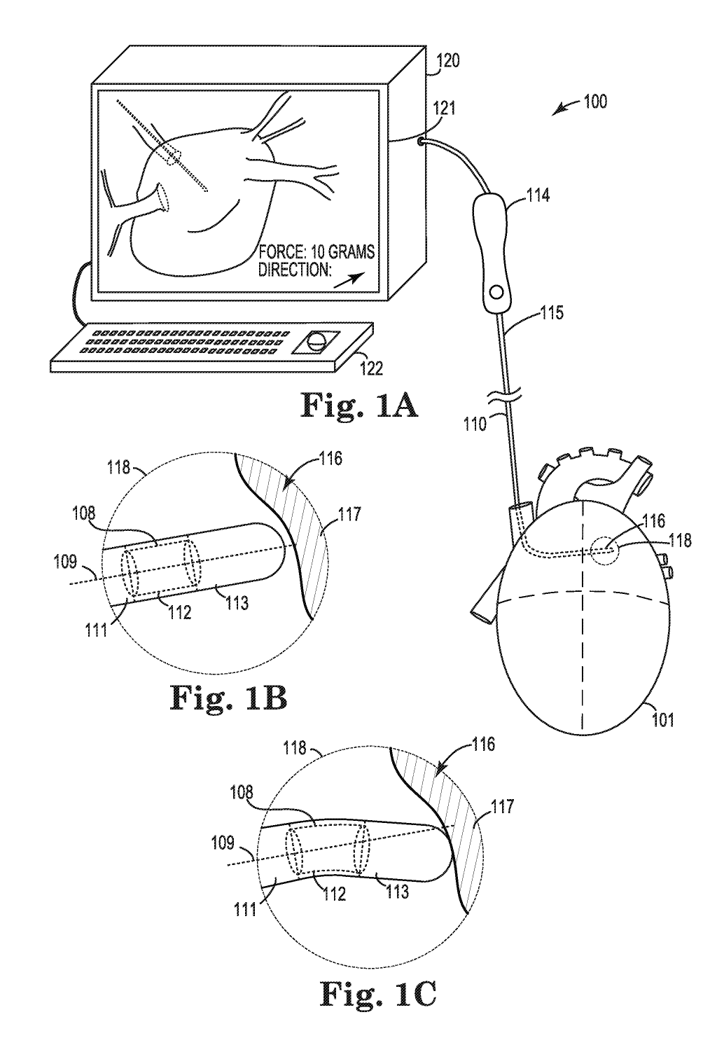

FIGS. 1A-C is an illustrative embodiment of a system 100 for sensing data from inside the body and/or delivering therapy. For example, the system 100 can be configured to map cardiac tissue and/or ablate the cardiac tissue, among other options. The system 100 includes a catheter 110 connected to a control unit 120 via handle 114. The catheter 110 can comprise an elongated tubular member having a proximal end 115 connected with the handle 114 and a distal end 116 configured to be introduced within a heart 101 or other area of the body. As shown in FIG. 1A, the distal end 116 of the catheter 110 is within the left atrium.

As shown in the window 118 of FIG. 1B, the distal end 116 of the catheter 110 includes a proximal segment 111, a spring segment 112, and a distal segment 113. The distal segment 113, or any other segment, can be in the form of an electrode configured for sensing electrical activity, such as electrical cardiac signals. Such an electrode (or other electrode on the catheter 110) can additionally or alternatively be used to deliver ablative energy to tissue.

The proximal segment 111, the spring segment 112, and the distal segment 113 can be coaxially aligned with each other in a base orientation as shown in FIG. 1B. Specifically, each of the proximal segment 111, the spring segment 112, and the distal segment 113 are coaxially aligned with a common longitudinal axis 109. The longitudinal axis 109 can extend through the radial center of each of the proximal segment 111, the spring segment 112, and the distal segment 113, and can extend through the radial center of the distal end 116 as a whole. In some embodiments, the coaxial alignment of the proximal segment 111 with the distal segment 113 can correspond to the base orientation. As shown, the distal end 116, at least along the proximal segment 111, the spring segment 112, and the distal segment 113, extends straight. In some embodiments, this straight arrangement of the proximal segment 111, the spring segment 112, and the distal segment 113 can correspond to the base orientation. The proximal segment 111, the spring segment 112, and the distal segment 113 can be mechanically biased to assume the base orientation.

A force measurement assembly 108 can reside within the distal end 116 of the catheter 110. The force measurement assembly 108 can extend from the proximal segment 111, through the spring segment 112, to the distal segment 113. While a single force measurement assembly 108 is shown in FIGS. 1B-C, a plurality of force measurement assemblies can be provided and each can be configured in any manner as the force measurement assembly 108 as described herein. The force measurement assembly 108 can mechanically support the distal segment 113 relative to the proximal segment 111. For example, the force measurement assembly 108 can provide most or all of the mechanical support that holds the distal segment 113 in the base orientation with respect to the proximal segment 111. In some embodiments, it is the force measurement assembly 108 which can provide the resilient spring properties of the spring segment 112. A proximal end of the force measurement assembly 108 can be anchored in the proximal segment 111 while a distal end of the force measurement assembly 108 can be anchored within the distal segment 113. For example, the proximal end of the force measurement assembly 108 can be rigidly attached to material within the proximal segment 111 while the distal end of the force measurement assembly 108 can be rigidly attached to material within the distal segment 113. The force measurement assembly 108 can be generally elongated from the proximal segment 111 to the distal segment 113.

The catheter 110 includes force sensing capabilities. For example, the catheter 110 is configured to sense a force due to engagement with tissue 117. The distal segment 113 can be relatively rigid while segments proximal of the distal segment 113 can be relatively flexible. In particular, the spring segment 112 may be more flexible than the distal segment 113 and the proximal segment 111 such that when the distal end 116 of the catheter 110 engages tissue 117, the spring segment 112, as shown in FIG. 1C, bends. For example, the distal end 116 of the catheter 110 can be generally straight as shown in FIG. 1B. When the distal segment 113 engages tissue 117, the distal end 116 of the catheter 110 can bend at the spring segment 112 such that the distal segment 113 moves relative to the proximal segment 111. As shown in FIGS. 1B and 1C, the normal force from the tissue moves the distal segment 113 out of coaxial alignment (e.g., with respect to the longitudinal axis 109) with the proximal segment 111 while the spring segment 112 bends. As such, proximal segment 111 and the distal segment 113 may be stiff to not bend due to the force while the spring segment 112 may be less stiff and bend to accommodate the force exerted on the distal end 116 of the catheter 110.

The force measurement assembly 108, which extends through or around the spring segment 112, can be used to determine the magnitude and the direction of the force due to engagement with the tissue 117. As shown in FIG. 1C, the distal segment 113 has moved relative to the proximal segment 111, thereby straining the force measurement assembly 108. Specifically, the force measurement assembly 108 is shown to be bending relative to the base orientation of the force measurement assembly 108 shown in FIG. 1B. The force measurement assembly 108 can be configured to sense such bending. The bending can change one or more electrical properties of the force measurement assembly 108. As further discussed herein, the force measurement assembly 108 can include an LC circuit that changes in resonance frequency in proportion to the bending, the bending being proportional to the force per Hooke's law. As such, by measuring a change in resonance frequency of one sensor, a parameter of the force, such as a magnitude of the force, can be determined for one axis. Three LC circuit-based sensors can be provided within the force measurement assembly 108 to characterize the bending, and therefore the force, in three axes (X, Y, and Z) to determine parameters of the force, such as the magnitude and direction of the force in three-dimensional space. These and other aspects are further discussed herein.

The control unit 120 of the system 100 includes a display 121 (e.g., LCD) for displaying information. The control unit 120 further includes a user input 122 which can comprise one or more buttons, toggles, a track ball, a mouse, touchpad, or the like for receiving user input. The user input 122 can additionally or alternatively be located on the handle 114. The control unit 120 can contain control circuitry for performing the functions referenced herein. Some or all of the control circuitry can alternatively be located within the handle 114.

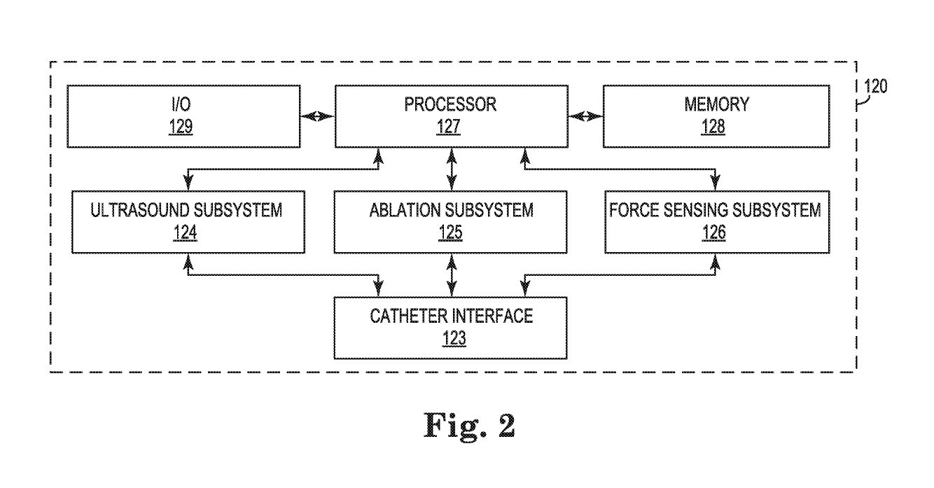

FIG. 2 illustrates a block diagram showing an example of control circuitry which can perform functions referenced herein. This or other control circuitry can be housed within control unit 120, which can comprise a single housing or multiple housings among which components are distributed. Control circuitry can additionally or alternatively be housed within the handle 114. The components of the control unit 120 can be powered by a power supply (not shown, but known in the art), which can supply electrical power to any of the components of the control unit 120 and the system 100. The power supply can plug into an electrical outlet and/or provide power from a battery, among other options.

The control unit 120 can include a catheter interface 123. The catheter interface 123 can include a plug which receives an electrical cable from the handle 114. The catheter 110 can include multiple conductors (not shown, but known in the art) to convey electrical signals between the distal end 116 and the proximal end 115 and further through handle 14 to the catheter interface 123. It is through the catheter interface 123 that the control unit 120 (and/or the handle 114, if control circuitry is included in the handle 114) can send electrical signals to any element within the catheter 110 and/or receive an electrical signal from any element within the catheter 110. The catheter interface 123 can conduct signals to or from any of the components of the control unit 120.

The control unit 120 can include an ultrasound subsystem 124 which includes components for operating the ultrasound functions of the system 100. While the illustrated example of control circuitry shown in FIG. 2 includes the ultrasound subsystem 124, it will be understood that not all embodiments may include ultrasound subsystem 124 or any circuitry for imaging tissue. The ultrasound subsystem 124 can include a signal generator configured to generate a signal for ultrasound transmission and signal processing components (e.g., a high pass filter) configured to filter and process reflected ultrasound signals as received by an ultrasound transducer in a sense mode and conducted to the ultrasound subsystem 124 through a conductor in the catheter 110. The ultrasound subsystem 124 can send signals to elements within the catheter 110 via the catheter interface 123 and/or receive signals from elements within the catheter 110 via the catheter interface 123.

The control unit 120 can include an ablation subsystem 125. The ablation subsystem 125 can include components for operating the ablation functions of the system 100. While the illustrated example of control circuitry shown in FIG. 2 includes the ablation subsystem 125, it will be understood that not all embodiments may include ablation subsystem 125 or any circuitry for generating an ablation therapy. The ablation subsystem 125 can include an ablation generator to provide different therapeutic outputs depending on the particular configuration (e.g., a high frequency alternating current signal in the case of radio frequency ablation to be output through one or more electrodes). Providing ablation energy to target sites is further described, for example, in U.S. Pat. No. 5,383,874 and U.S. Pat. No. 7,720,420, each of which is expressly incorporated herein by reference in its entirety for all purposes. The ablation subsystem 125 may support any other type of ablation therapy, such as microwave ablation. The ablation subsystem 125 can deliver signals or other type of ablation energy through the catheter interface 123 to the catheter 110.

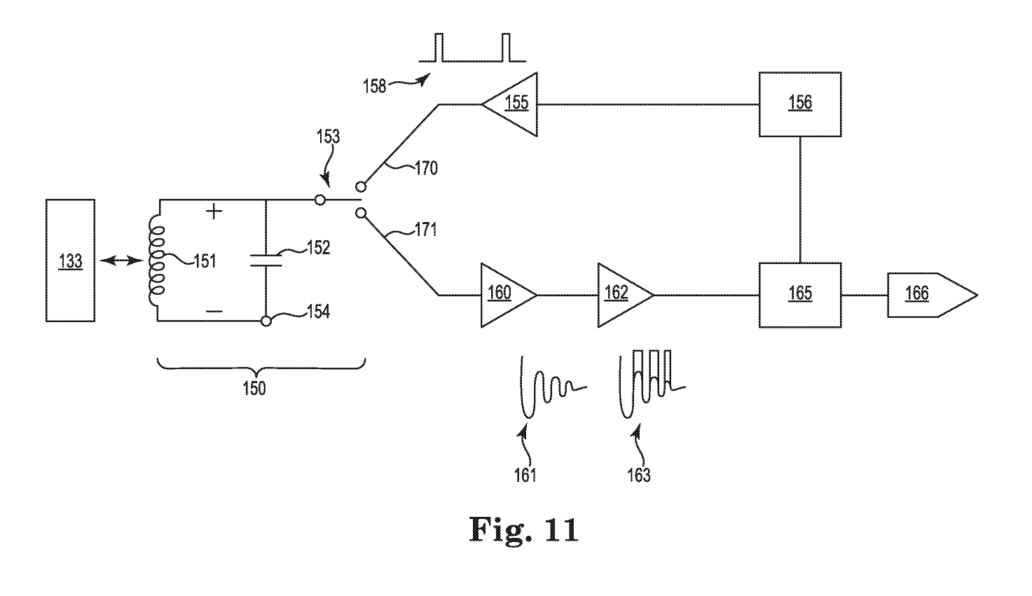

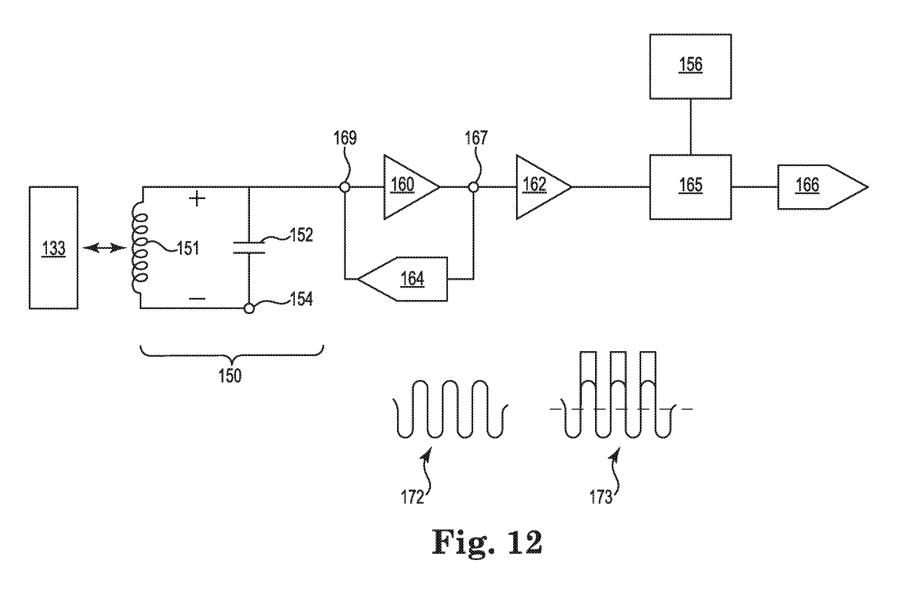

The control unit 120 can include a force sensing subsystem 126. The force sensing subsystem 126 can include components for measuring a force experienced by the catheter 110 via the force measurement assembly 108. The force sensing subsystem 126 can include some of the components shown in FIGS. 11 and 12. Such components can include signal processors, analog-to-digital converters, operational amplifiers, transistors, comparators, and/or any other circuitry for conditioning and measuring one or more signals. The force sensing subsystem 126 can send signals to elements within the catheter 110 via the catheter interface 123 and/or receive signals from elements within the catheter 110 via the catheter interface 123.

Each of the ultrasound subsystem 124, the ablation subsystem 125, and the force sensing subsystem 126 can send signals to, and receive signals from, the processor 127. The processor 127 can be any type of processor for executing computer functions. For example, the processor 127 can execute program instructions stored within the memory 128 to carry out any function referenced herein, such as determine the magnitude and direction of a force experienced by the catheter 110.

The control unit 120 further includes an input/output subsystem 129 which can support user input and output functionality. For example, the input/output subsystem 129 may support the display 121 to display any information referenced herein, such as a graphic representation of tissue, the catheter 110, and a magnitude and direction of the force experienced by the catheter 110, among other options. Input/output subsystem 129 can log key and/or other input entries via the user input 122 and route the entries to other circuitry.

A single processor 127, or multiple processors, can perform the functions of one or more subsystems, and, as such, the subsystems may share control circuitry. Although different subsystems are presented herein, circuitry may be divided between a greater or lesser number of subsystems, which may be housed separately or together. In various embodiments, circuitry is not distributed between subsystems, but rather is provided as a unified computing system. Whether distributed or unified, the components can be electrically connected to coordinate and share resources to carry out functions.

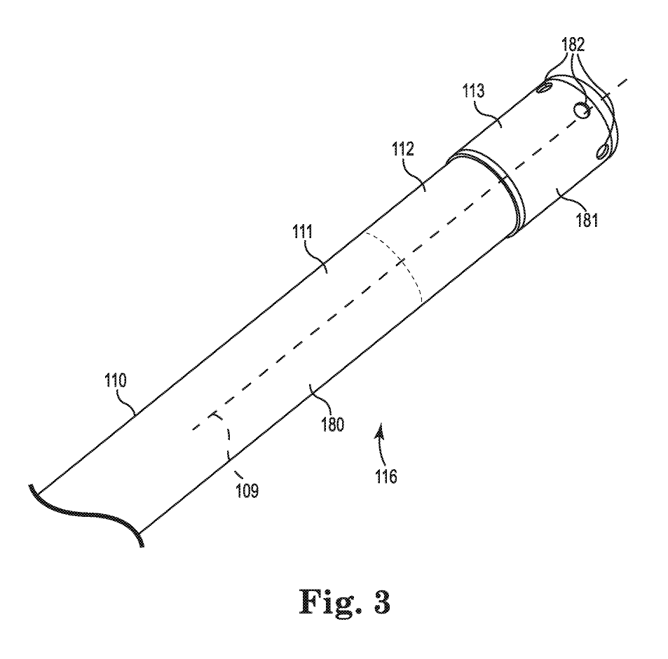

FIG. 3 illustrates a detailed view of a distal end 116 of a catheter 110. FIG. 3 shows a catheter shaft 180. The catheter shaft 180 can extend from the distal segment 113 to the handle 114, and thus can define an exterior surface of the catheter 110 along the spring segment 112, the proximal segment 111, and further proximally to the proximal end 115. The catheter shaft 180 can be a polymeric tube formed from various polymers, such as polyurethane, polyamide, polyether block amide, silicone, and/or other materials. In some embodiments, the catheter shaft 180 may be relatively flexible, and at least along the spring segment 112 may not provide any material mechanical support to the distal segment 113 (e.g., facilitated by thinning of the wall of the catheter shaft 180 along the spring segment 112). Alternatively, the catheter shaft 180 may provide structural mechanical support to the distal segment 113 relative to the spring segment 112 and the proximal segment 111.

As shown, the proximal segment 111 can be proximal and adjacent to the spring segment 112. The length of the proximal segment 111 can vary between different embodiments, and can be five millimeters to five centimeters, although different lengths are also possible. The length of the spring segment 112 can also vary between different embodiments, and can be dependent on the length of the force measurement assembly 108 as a whole or specifically on a spring element within the force measurement assembly 108. The spring segment 112 is adjacent to the distal segment 113. As shown in FIG. 3, the distal segment 113 can be defined by an electrode 181. The electrode 181 can be an ablation electrode. In some other embodiments, the distal segment 113 may not be an electrode. The electrode 181 can be in a shell form which can contain other components. The electrode 181 can include a plurality of ports 182. One or more ultrasonic transducers, housed within the electrode 181, can transmit and receive signals through the ports 182 or through additional dedicated ports in the tip shell. Additionally, or in place of the transducers, one or more miniature electrodes (not shown) may be incorporated into the tip shell assembly.

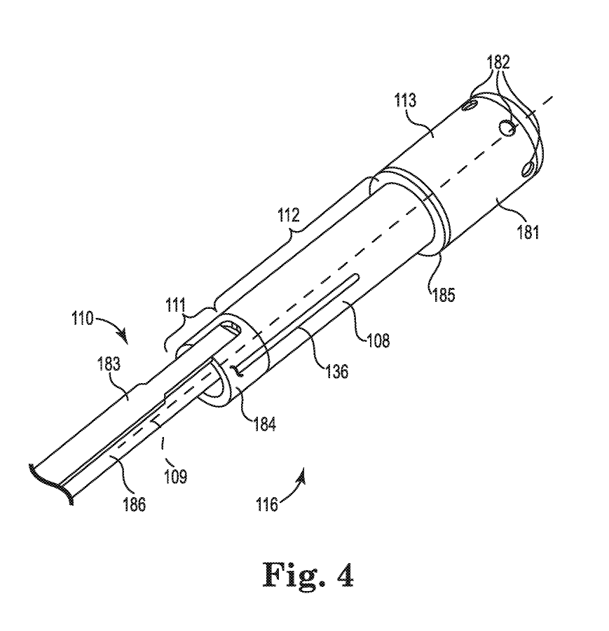

FIG. 4 shows the catheter 110 after the removal of the catheter shaft 180 to expose various components that underlie the catheter shaft 180. The removal of the catheter shaft 180 exposes structural and force sensing components. The components can include a proximal hub 184, a distal hub 185, and a force measurement assembly 108 that bridges between the proximal hub 184 and the distal hub 185. The proximal hub 184 and the distal hub 185 can be respective rings to which the force measurement assembly 108 is attached proximally and distally, respectively. One or both of the proximal hub 184 and the distal hub 185 can be formed from electrically insulative material, such as polymer (e.g., polyethylene or polyether ether ketone); an electrically conductive material, such as a metal (e.g., stainless steel, titanium, or aluminum); and/or a composite or ceramic material.

The proximal hub 184 and the distal hub 185 can be coaxially aligned with respect to the longitudinal axis 109. For example, the longitudinal axis 109 can extend through the respective radial centers of each of the proximal hub 184 and the distal hub 185. One or more inner tubes 186 (one shown) can extend through the catheter 110 (e.g., to the handle 114), through the proximal hub 184 and the distal hub 185. The inner tube 186 can include one or more lumens within which one or more conductors can extend from the proximal end 115 to the distal segment 113, such as for connecting with one or more electrical elements (e.g., ultrasound transducer, electrode, the force measurement assembly 108, or other component). Coolant fluid can additionally or alternatively be routed through the inner tube 186, or through an additional inner tube 186. In various embodiments, the catheter 110 is open irrigated (e.g., through the plurality of ports 182) to allow the coolant fluid to flow out of the distal segment 113. Various other embodiments concern a non-irrigated catheter 110.

A tether 183 can attach to a proximal end of the proximal hub 184. The tether 183 can attach to a deflection mechanism within a handle to cause deflection of the distal end 116. A knob, slider, or plunger on a handle may be used to create tension or slack in the tether 183.

As shown in FIG. 4, the spring segment 112 can extend from a distal edge of the proximal hub 184 to a proximal edge of the distal hub 185. As such, the proximal hub 184 can be part of, and may even define the length of, the proximal segment 111. Likewise, the distal hub 185 can be part of the distal segment 113. The proximal hub 184 and the distal hub 185 can be stiffer than the force measurement assembly 108 such that a force directed on the distal segment 113 causes the distal end 116 to bend along the force measurement assembly 108 (the spring segment 112 specifically) rather than along the distal segment 113 or the proximal segment 111. Even within the force measurement assembly 108, the bending may be isolated to an expanse of a spring element further discussed herein. The distal segment 113 may be mechanically maintained in a base orientation with respect to the longitudinal axis 109 mostly or entirely by the force measurement assembly 108 (e.g., wherein all other components contribute negligible or no mechanical support of the distal segment 113 relative the proximal segment 111). In some other embodiments, other elements can provide mechanical support to the distal segment 113, such as the catheter shaft 180 and/or internal struts.

The proximal end of the force measurement assembly 108 can be attached to the distal end of the proximal hub 184, such as by a press fit feature, an adhesive (e.g., epoxy), welding, staking, pinning, and/or riveting. The distal end of the force measurement assembly 108 can be attached to the proximal end of the distal hub 185, such as by a press fit feature, an adhesive (e.g., epoxy), welding, staking, pinning, and/or riveting. It is noted that each of the proximal hub 184, the force measurement assembly 108, and the distal hub 185 can include coaxial lumens that allow the inner tube 186 or other element to extend within the lumens of the proximal hub 184, the force measurement assembly 108, and the distal hub 185 to the distal segment 113 and the electrode 181. The conductors (not illustrated) can be routed through the inner tube 186 from one or more elements along the distal segment 113 to a proximal end of the catheter 110 for delivering signals to and/or from control circuitry. The conductors can be copper wires insulated by a polymer coating, or can be similar conductive elements.

A tail 136 extends from the force measurement assembly 108 in a proximal direction. The tail 136 can be a plurality of individual insulated conductors, or part of a printed circuit board that includes a plurality of conductors. The tail 136 can extend further proximally to the proximal end 115 of the catheter 110 to electrically connect with the control circuitry. The tail 136 can alternatively be routed within the lumens within the force measurement assembly 108, the proximal hub 184, and/or the inner tube 186. However, as shown, the tail 136 extends within the catheter shaft 180 but outside of the force measurement assembly 108, the proximal hub 184, and the catheter shaft 180.

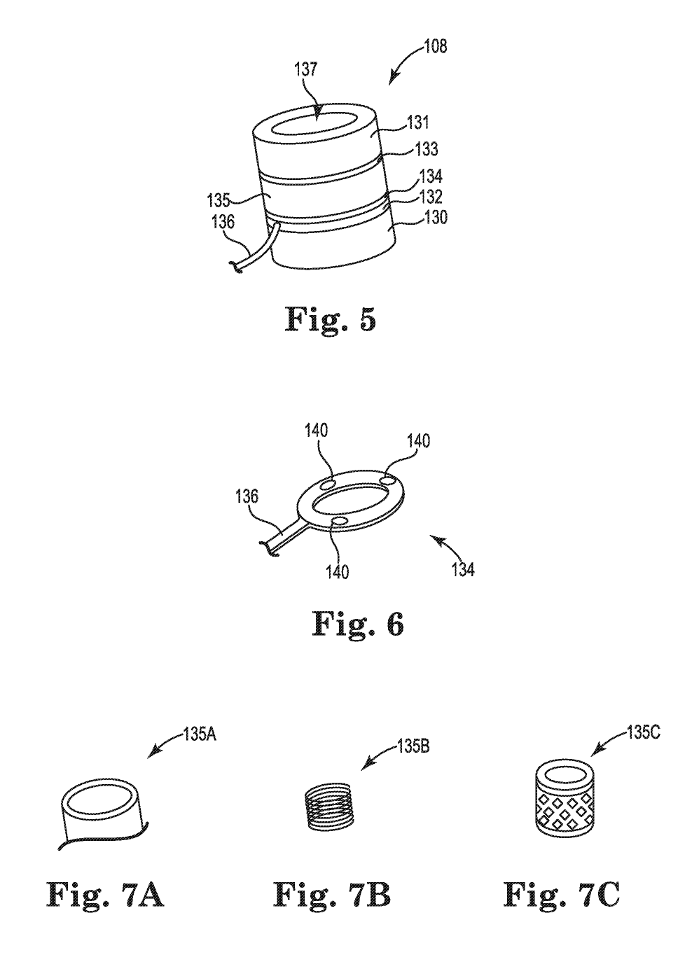

FIG. 5 shows a perspective view of the force measurement assembly 108 isolated from the other components of the catheter 110. The force measurement assembly 108 is in a generally tubular shape and includes a lumen 137 that extends the full-length of the force measurement assembly 108. As such, in some embodiments, all sensory components of the force measurement assembly 108 can be contained in a tubular wall of the force measurement assembly 108.

The force measurement assembly 108 is shown to include a proximal support 130. A proximal end of the proximal support 130 can attach to a distal end of the proximal hub 184. In some embodiments, the proximal hub 184 and the proximal support 130 are unified into a single element, but in the present example are shown as separate elements. The force measurement assembly 108 is shown to include a distal support 131. A distal end of the distal support 131 can attach to a proximal end of the distal hub 185. In some embodiments, the distal hub 185 and the distal support 131 are unified into a single element, but in the present example are shown as separate elements. The proximal support 130 and the distal support 131 can each be formed from polymer (e.g., polyethylene, polyether ether ketone, or polyoxymethylene), a metal (e.g., stainless steel, aluminum, or nitinol), or a ceramic. In some embodiments, the proximal support 130 and the distal support 131 are each configured to not compress when the force is applied to the catheter 110. In other words, the proximal support 130 and the distal support 131 (as well as the proximal hub 184 and the distal hub 185) pass all forces through themselves and do not strain under stress.

The force measurement assembly 108 is further shown to include a proximal mass 132 and a distal mass 133. The proximal mass 132 can be attached to a distal end of the proximal support 130. The distal mass 133 can be attached to a proximal end of the distal support 131. Such attachments can be made with an adhesive such as epoxy adhesive, or by welding, staking, pinning, or riveting. Each of the masses 132, 133 can be in the shape of a ring that is consistent with the tubular profile of the force measurement assembly 108 (e.g., by having the same inner diameter and outer diameter of the tubular structure of the force measurement assembly 108). While each of the proximal mass 132 and the distal mass 133 are shown as respective unitary elements (in this case, rings), each mass could be comprised of the same type of material but comprise several distinct masses supported in a ring structure. For example, a plurality of masses can be embedded within the distal end of the proximal support 130, the plurality of masses arrayed circumferentially about the lumen 137. A plurality masses can be embedded within the proximal end of the distal support 131, the plurality masses arrayed circumferentially about the lumen 137.

The proximal mass 132 and the distal mass 133 can be formed from material having high magnetic permeability, such as ferrite. In some embodiments, the high magnetic permeability material forming proximal mass 132 and the distal mass 133 has a relative permeability greater than 1500. The proximal mass 132 and the distal mass 133 can be passive, such that each is not electrically activated by current during any phase of force sensing. In some cases, each of the proximal mass 132 and the distal mass 133 do not emit an electromagnetic field. However, a mass material having a high magnetic permeability can affect the emitted electromagnetic field of other elements, as further shown herein.

The force measurement assembly 108 is further shown to include a sensor support 134. The sensor support 134 can be a printed circuit board. Such a printed circuit board can comprise a flat, flexible sheet of a base polymer layer (e.g., polyimide), a trace layer (e.g., a flat copper, gold, silver, or nickel conductor), and a polymer cover coat (e.g., polyamide) over the trace layer. In some embodiments, a greater number of layers can be built from these components, such as overlapping but electrically isolated trace layers.

FIG. 6 shows a perspective view of the sensor support 134. The sensor support 134 includes a plurality of sensors 140. The plurality of sensors 140 can be embedded within or mounted upon the sensor support 134. Three sensors 140 are shown circumferentially arrayed about the lumen 137. The use of three sensors 140 can be advantageous for characterizing forces in three dimensions (e.g., along X, Y, and Z axes). It is noted that the plurality of sensors 140 are arrayed at 120° about the circumference of the sensor support 134. In other embodiments, more than three sensors 140 may employed, and the plurality of sensors 140 may be with disposed in different uniform and non-uniform circumferential arrangements. The sensor support 134 includes a tail 136 which can be made from the same printed circuit board material as the rest of the sensor support 134. The tail 136 can include a plurality of conductors that electrically connect, respectively, with the plurality of sensors and extend to a proximal end of the catheter 110 for connecting with different channels of control circuitry.

As shown in FIG. 5, the force measurement assembly 108 further comprises a spring element 135. The spring element 135 is positioned between the sensor support 134 and the distal mass 133. The spring element 135 can be attached to any of the components of the force measurement assembly 108, such as the proximal support 130, the distal support 131, the proximal mass 132, the distal mass 133, and/or the sensor support 134. In some embodiments, the spring element 135 may connect the distal hub 185 to the proximal hub 184, thereby controlling the relative displacement between the sensor support 134 and the distal mass 133. In some embodiments, the force measurement assembly 108 only bends along the spring element 135 such that the force measurement assembly 108 does not bend along the proximal support 130, the distal support 131, the proximal mass 132, the distal mass 133, and the sensor support 134. The spring element 135 can be configured to permit bending of the force measurement assembly 108 (and the distal end 116 of the catheter 110 as shown in FIG. 1C) while resiliently returning the force measurement assembly 108 (and the distal end 116 of the catheter 110) to the base orientation shown in FIG. 1B after removal of the force. In some embodiments, the force measurement assembly 108 may provide most or all of the mechanical support that holds the distal segment 113 in the base orientation with respect to the proximal segment 111 and resiliently returns the distal segment 113 to the base orientation with respect to the proximal segment 111 after removal of the force. In some of these embodiments, the spring element 135 may provide most or all of the mechanical support that holds the distal segment 113 in the base orientation with respect to the proximal segment 111 and resiliently returns the distal segment 113 to the base orientation with respect to the proximal segment 111 after removal of the force. However, in some other embodiments, the catheter shaft 180 may provide some or most of the mechanical support for the distal segment 113 relative to the proximal segment 111.

Each layer of the force measurement assembly 108 can be attached to the adjacent layers of the force measurement assembly 108 by adhesive (e.g., epoxy) and/or a tube or layer of polymer wrapped around the circumference of the force measurement assembly 108 to encase the force measurement assembly 108 and secure the layers. In other embodiments, some or all of the layers may be mechanically attached to one another by pinning or staking.

The spring element 135 can take different forms. FIG. 7A shows a perspective view of one form. The spring element 135A of FIG. 7A can be an elastomeric member. The elastomeric member can have the same inner diameter and/or outer diameter as any other components of the force measurement assembly 108. The spring element 135A can be formed from rubber, silicone, or other elastomeric polymer that can be compressed or stretched due to a force and then can resiliently return to its original form after removal of the force. FIG. 7B shows a spring element 135B as an alternative design to that of FIG. 7A. Specifically, the spring element 135B is a coiled metal spring, which can be formed from stainless steel or nitinol, for example. The coiled metal spring can have the same inner diameter and/or outer diameter as any other components of the force measurement assembly 108. FIG. 7C shows a spring element 135C as an alternative design to that of FIGS. 7A and 7B. Specifically, the spring element 135C is a cut, etched, or formed metallic tubular element, which can be made from, for example, stainless steel or nitinol. The metallic tubular element can have the same inner diameter and/or outer diameter as any other components of the force measurement assembly 108.

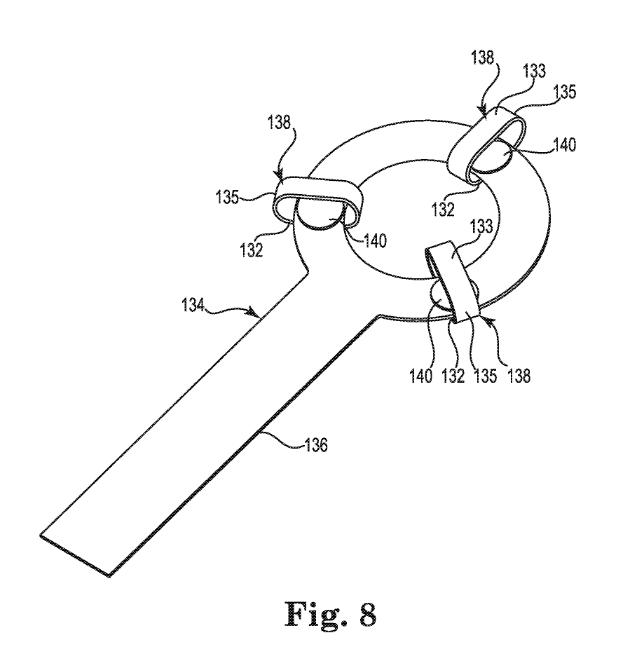

FIG. 8 shows a perspective view of a portion of another embodiment of the force measurement assembly 108. FIG. 8 shows a portion of the force measurement assembly 108, including the proximal mass 132, the distal mass 133, the sensor support 134, and the spring element 135. The proximal mass 132, the distal mass 133, and the spring element 135 are formed as a combined element 138. The curved portion of the combined element 138 connecting the proximal mass 132 to the distal mass 133 forms the spring element 135. Three of the combined elements 138 can be circumferentially arrayed around the sensor support 134, with one at each of the sensors 140. In this way, the combined element 138 may capture and contain most of the induced magnetic flux at the sensor 140, while also providing the resiliency needed to allow for a change in the resonance frequency of the sensor 140.

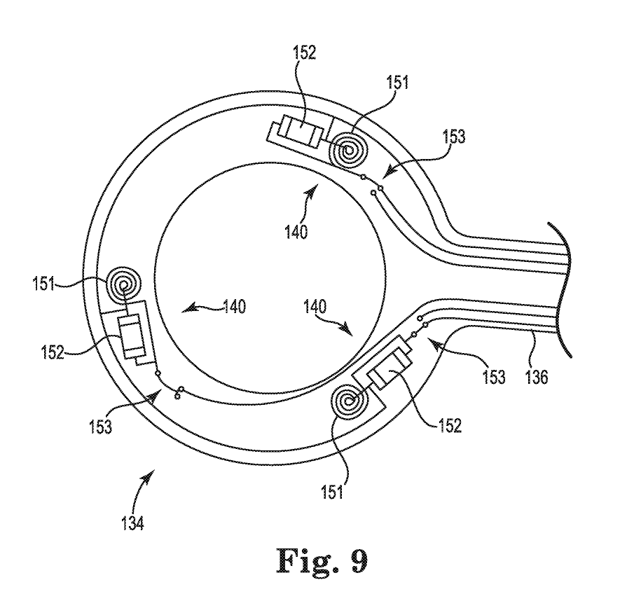

FIG. 9 shows an overhead sectional view of the sensor support 134. The view of FIG. 9 shows circuit components as exposed, wherein the circuit components may otherwise be normally covered by insulation. Each sensor 140 comprises an LC circuit composed of an inductor 151 and a capacitor 152 connected electrically in parallel to each other. Each sensor 140 further includes a switch 153 which can change an electrical connection to the LC circuit from an exciter channel to a measurement channel, as further discussed herein. While the switches 153 are included on the sensor support 134, in some other embodiments the switches 153 are located elsewhere within the catheter 110, handle 114, or control unit 120. It is noted that switches 153 can be replaced with transformers in various embodiments.

The inductors 151 can be flat conductor spirals disposed within the printed circuit board of the sensor support 134. As such, the inductors 151 do not comprise helical coil windings, and are therefore more compact. The electromagnetic field generated by each flat conductor spiral may be particularly small, but such a small field may be advantageous because it is easier to fill this small field with high magnetic permeability material (as further discussed herein) and because the small field minimizes the potential sources of noise which may otherwise influence the field.

In some other embodiments, the inductors 151 can include helical windings. While each sensor 140 is shown as including one inductor 151, two (or more) identical inductors can be provided for each sensor 140 with the multiple inductors 151 serially electrically connected. When multiple flat conductor spirals are used, the multiple flat conductor spirals can be stacked on top of each other while still being within the printed circuit board of the sensor support 134. When connected in series, the inductances of the stacked flat conductor spirals are additive. Alternatively, the multiple inductors can be placed side-by-side. In other embodiments, the inductors 151 can include an externally wound inductive coil component that is attached to the sensor support 134 and electrically connected by, for example, solder or conductive epoxy.

The sensors 140 can operate by exciting each LC circuit with a pulse of energy. For example the switch 153 can connect to a conductor which can transport a pulse of electrical energy to the LC circuit. Once energized from the pulse, the LC circuit will oscillate for a brief period of time. In particular, the LC circuit will oscillate at its resonance frequency. The resonance frequency of the LC circuit is governed by the relationship shown in Equation 1:

f=1/[2π√(LC)] Equation 1:

In Equation 1, the resonance frequency (f) of oscillation is a function of an inductance (L) and a capacitance (C). As will be explained further herein, the inductance of the LC circuit may vary based on the proximity of the distal mass 133 of high magnetic permeability material to the inductor 151, wherein the proximity is variable based on the force applied to the catheter 110.

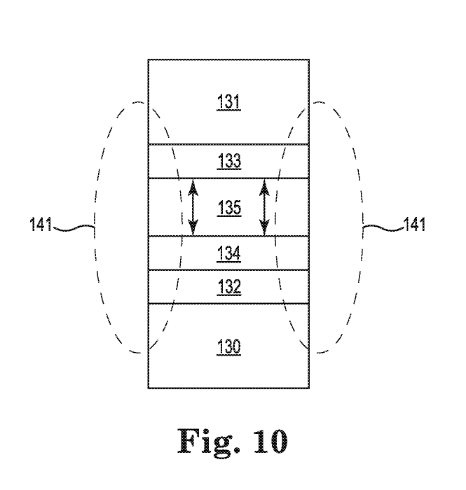

FIG. 10 is a schematic view of a portion of the force measurement assembly 108. As discussed previously, the sensor support 134 can include sensors 140 which themselves include inductors 151. During oscillation of the LC circuit following excitation from a pulse, the inductors 151 temporarily create magnetic fields 141. Within the envelopes of the magnetic fields 141 are the proximal mass 132 and the distal mass 133, each formed of high magnetic permeability material. The presence of the high magnetic permeability material within the magnetic fields 141 increases the strength of these fields relative to other materials and/or air. As shown in FIG. 9, the distance from the sensor support 134 (containing the inductors 151) and the distal mass 133 of high magnetic permeability material is variable. The distance can be made shorter by the force placed on the distal segment 113 of the catheter 110 compressing the spring element 135. Some forces may compress one portion of the spring element 135 and elongate another portion of the spring element 135, thereby bringing the distal mass 133 of high magnetic permeability material closer to one or more of the sensors 140 but further away from one or more of the other sensors 140. In some other cases, the force may compress different circumferential sections of the spring element 135 to different degrees, such that one or more sensors 140 is brought closer to the distal mass 133 than other sensors 140. In any case, increasing or decreasing the amount of the distal mass 133 of high magnetic permeability material within the fields 141 changes the inductance of the LC circuits, which according to Equation 1 changes the resonance frequency of the LC circuits. As such, the change in resonance frequency is proportional to the displacement of the distal mass 133 of high magnetic permeability material relative to the sensor support 134. The spring element 135 provides predictable resistance to movement of the distal mass 133 relative to the sensor support 134, a relationship governed by Hooke's law (force=−kx, wherein k is a spring constant and x is displacement). Therefore, a known displacement can be correlated to a force value by using a spring constant value for the spring element 135. The spring constant can be determined experimentally for each unit as part of a calibration step or can be predetermined for each type of spring element 135 and stored in a memory, such as memory 128 described above in reference to FIG. 2. In summary, the displacement of the distal mass 133 relative to the sensor 140 is proportional to the force placed on catheter 110. The displacement of the distal mass 133 further results in a change in the inductance (L) of the associated inductor 151, which results in a change in the resonance frequency of the sensor 140 per Equation 1. Multiple resonance frequency changes of multiple spatially distributed sensors 140 can indicate the magnitude and direction of the force in three dimensions. Such a force-frequency relationship is represented by Equation 2:

F=−k [symbol] (1[(2πf)²C]), Equation 2:

wherein [symbol] is the function relating resonance frequency (f) to the displacement (x) of the distal mass 133.

It is noted that operation of the sensors 140 do not involve any of the sensors sensing a magnetic field broadcast from a remote element within the catheter 110. Rather, each sensor 140 generates its own magnetic field and changes in the magnetic field, due to increasing or decreasing amounts of passive high magnetic permeability material within the magnetic field, are sensed. This minimizes the influence of noise on the sensor 140. Moreover, each of the proximal mass 132 and the distal mass 133 of high magnetic permeability material help to shield the sensors 140 from electromagnetic noise.

In some embodiments, the resonance frequency can be identified for each sensor 140 by pulsing each LC circuit at a sampling rate ranging from, for example 10 to 1000 times per second, with each pulse causing the LC circuit to oscillate at its respective resonance frequency (f) (e.g., 1 MHz to 10 MHz). In other embodiments, the resonance frequency can be identified for each sensor 140 from a continuous waveform at the resonant frequency of the LC circuit by re-energizing the LC circuit on each cycle by injecting electrical energy into the LC circuit at the correct phase of the waveform.

FIG. 11 shows circuitry which can support pulsing each sensor 140 at a sampling rate and then measuring its resonance frequency. FIG. 11 shows the distal mass 133 having a variable distance from the inductor 151. Inductor 151 is part of an LC circuit 150 and electrically parallel with the capacitor 152. A switch 153 can electrically connect the LC circuit 150 with an excitation branch 170 or a measurement branch 171. The excitation branch 170 includes a driver 155 which can generate pulses of electrical energy as shown in excitation signal 158. The timing of pulses can be determined in part by clock 156. The switch 153 can alternatively connect the LC circuit 150 to the measurement branch 171. It is noted that the LC circuit 150 includes a node 154 which can be used as a reference for making voltage measurements across the LC circuit 150 together with the measurement branch 171. The measurement branch 171 includes an amplifier 160 which can output sinusoidal signal 161 indicative of the oscillation in the LC circuit 150 due to one excitation pulse from driver 155. A threshold detector 162 can be used to identify the period of a cycle of the sinusoidal signal 161 as shown by signal 163. A digital comparator 165 can identify a preset number of oscillation cycles from the signal 163. The identification of the cycles can be facilitated by input from the clock 156 which can indicate, among other things, the timing of the excitation pulses of the excitation signal 158. Based on the clock 156 and the timing of the oscillation cycles, a frequency of oscillation measured from the measurement branch 171 can be determined for each excitation cycle output (e.g., corresponding to each pulse) by the excitation branch 170. Thus, for each excitation pulse, a resonance frequency can be measured. The resonance frequency can be sent from an output 166 to the processor 127 (FIG. 2) or to a processor within the force sensing subsystem 126 to compare to recent resonance frequency values for the same LC circuit 150 to detect a change in frequency over time (e.g., relative to a baseline indicating no force), which can be related to a change in force.

FIG. 12 shows circuitry which can support continuously oscillating each sensor 140 to produce a continuous sinusoidal output and then measuring its resonance frequency. Unlike the pulsed embodiment described above in reference to FIG. 11, this embodiment does not need the switch 153 in each sensor 140 because it does not have separate excitation and measurement branches. FIG. 12 shows the distal mass 133 having a variable distance from the inductor 151. Inductor 151 is part of an LC circuit 150 and electrically parallel with the capacitor 152. The LC circuit 150 can be electrically connected to an amplifier 160. A positive feedback device 164 can connect a node 167 at the output of the amplifier 160 to a node 169 at the input of the amplifier 160 to re-energize the LC circuit 150 such that the amplifier 160 outputs sinusoidal signal 172 indicative of the oscillation in the LC circuit 150. A threshold detector 162 can be used to identify the period of a cycle of the sinusoidal signal 172 as shown by signal 173. A digital comparator 165 can identify a preset number of oscillation cycles from the signal 173. The identification of the cycles can be facilitated by input from the clock 156. Based on the clock 156 and the timing of the oscillation cycles, a frequency of oscillation can be determined. The resonance frequency can be sent from an output 166 to the processor 127 (FIG. 2) or to a processor within the force sensing subsystem 126 (FIG. 2) to compare to recent resonance frequency values for the same LC circuit 150 to detect a change in frequency over time (e.g., relative to a baseline indicating no force), which can be related to a change in force.

The circuits shown in FIGS. 11 and 12 can be duplicated but without a variable distance mass of high magnetic permeability material. Such a duplicate circuit may function as a control sensor to cancel out noise. For example, the control sensor can be located proximally of the proximal mass 132. Changes in the resonance frequency of the control sensor can be subtracted from changes in resonance frequency of the other sensors 140 to cancel out environmental noise interference or variations due to changes in temperature.

As shown in FIG. 6, the sensors 140 are circumferentially arrayed about the sensor support 134. If the force exerted on the distal segment 113 of the catheter 110 is coaxial with the longitudinal axis 109, then each of the sensors 140 will indicate equal amounts of resonance frequency change (and therefore equal amount of movement relative to the distal mass 133). Based on these equal changes, the control circuitry can determine a magnitude and direction of the force. In some embodiments, the displacement relative to each sensor 140 can be calculated based on the change in resonance frequency for that sensor 140 and the change in dimension can then be used to calculate force based on Hooke's law. Alternatively, the force can be calculated using a combined equation that incorporates Hooke's law and the equation for determining the displacement (x) from the resonance frequency (f) for an LC circuit (e.g., Equation 2). Returning to the above example in which the changes in resonance frequency and displacements are respectively equal for each of the sensors 140, the control circuitry can determine that the force is coaxial with the longitudinal axis 109.

If the force is not coaxial with the longitudinal axis 109, then distal segment 113 will tend to curl or shift radially away from the force with respect to the proximal segment 111. In such cases, the sensors 140 will indicate different changes in resonance frequency. For example, all sensors 140 may indicate different levels of decreasing resonance frequency. In some cases, one or more sensors 140 may indicate an increasing resonance frequency, while one or more other sensors 140 may indicate a decreasing resonance frequency. Which sensors 140 indicate increasing resonance frequency and which sensors indicate decreasing resonance frequency depends on the direction of the force and the off-axis movement of the distal segment 113 relative to the proximal segment 111. If the force had a different direction, a different one or more of the sensors 140 will indicate an increasing resonance frequency while another will indicate a different level of resonance frequency change or a decrease in resonance frequency. Generally, the one or more sensors 140 that indicate a decrease in resonance frequency indicate the direction from which the force is coming while the one or more sensors 140 that indicate an increase in resonance frequency indicate the opposite direction (in which the force is pointed or going). Based on this, the direction (e.g., unit vector) of the force can be determined by the control circuitry. It is noted that baseline resonance frequency values can be determined for each of the sensors 140 based on a number of consecutive constant resonance frequency values. Deviation from this baseline indicates a force acting on the catheter.

Once assembled, the catheter 110 may undergo a calibration step, either at a factory or just before use by a physician. In such a step, a plurality of forces of known magnitude and direction can be placed, in sequence, on the distal segment 113 to move the distal segment 113 relative to the proximal segment 111, while the sensors 140 output signals or otherwise exhibit changes in resonance frequency indicative of the strain of the spring element 135. A table can be generated indicating a separate entry for each calibration force value (magnitude and direction). Thereafter, a force of unknown magnitude and/or direction can be analyzed by comparing signals output from the sensors 140 to the values of the table to identify the best match. An algorithm can identify which entry from the calibration data has three (or other number depending on the number of sensors 140) change-in-resonance frequency values best matching the current change-in-resonance frequency values. The magnitude and direction of the known force from the calibration step can be indicated as the magnitude and direction currently being experienced. In some cases, a mathematical relationship can be generated based on the linearity of Hooke's law, wherein a limited number of calibration steps are performed to determine the change-in-resonance frequency, or other parameter, and interpolation and/or extrapolation can be computed based on these calibration values. For example, the spring constant can be determined for the spring element 135 such that a subsequent amount of change in separation distance between a sensor 140 and the distal mass 133 can be multiplied by the spring constant to determine the magnitude of the force acting on the distal segment 113. The changes in separation distance for multiple sensors 140 can be factored for determining an overall magnitude and direction for the force.

The magnitude can be represented in grams or another measure of force. The magnitude can be presented as a running line graph that moves over time to show new and recent force values. The direction can be represented as a unit vector in a three dimensional reference frame (e.g., relative to an X, Y, and Z axes coordinate system). In some embodiments, a three dimensional mapping function can be used to track the three dimensional position of the distal end 116 of the catheter 110 in the three dimensional reference frame. Magnetic fields can be created outside of the patient and sensed by a sensor that is sensitive to magnetic fields within distal end 116 of the catheter 110 to determine the three dimensional position of the distal end 116 of the catheter 110 in the three dimensional reference frame. The direction can be represented relative to the distal end 116 of the catheter 110. For example, a line projecting to, or from, the distal segment 113 can represent the direction of the force relative to the distal segment 113. Such representations can be made on a display as discussed herein.

In some embodiments, the magnitude and direction of the force that are indicated to the user indicate the magnitude and the direction of a force that acts on the distal segment 113. This force typically results from the distal segment 113 pushing against tissue. Therefore, the force acting on the distal segment 113 may be a normal force resulting from the force that the distal segment 113 exerts on the tissue. In some embodiments, it is the force acting on the distal segment 113 that is calculated and represented to a user. Additionally or alternatively, it is the force that the distal segment 113 applies to the tissue that is calculated and represented to the user.

The magnitude and direction of the force can be used for navigation by providing an indicator when the catheter encounters tissue and/or for assessing the lesioning of tissue by determining the degree of contact between the lesioning element and the tissue, among other options. In some embodiments, a force under 10 grams is suboptimal for lesioning tissue (e.g., by being too small) while a force over 40 grams is likewise suboptimal for lesioning tissue (e.g., by being too large). Therefore, a window between 10 and 40 grams may be ideal for lesioning tissue and the output of the force during lesioning may provide feedback to the user to allow the user to stay within this window. Of course, other force ranges that are ideal for lesioning may be used.

The techniques described in this disclosure, including those attributed to a system, control unit, control circuitry, processor, or various constituent components, may be implemented wholly or at least in part, in hardware, software, firmware or any combination thereof. A processor, as used herein, refers to any number and/or combination of a microprocessor, a digital signal processor (DSP), an application specific integrated circuit (ASIC), a field-programmable gate array (FPGA), microcontroller, discrete logic circuitry, processing chip, gate arrays, and/or any other equivalent integrated or discrete logic circuitry. As part of the control circuitry, at least one of the foregoing logic circuitry can be used, alone or in combination with other circuitry, such as memory or other physical medium for storing instructions, can be used to carry about specified functions (e.g., a processor and memory having stored program instructions executable by the processor for determining a magnitude and a direction of a force exerted on a catheter based on a change in resonance of at least one sensor circuit within the catheter). The functions referenced herein may be embodied as firmware, hardware, software or any combination thereof as part of control circuitry specifically configured (e.g., with programming) to carry out those functions, such as a means for performing the functions referenced herein. The steps described herein may be performed by a single processing component or multiple processing components, the latter of which may be distributed among different coordinating devices. In this way, control circuitry may be distributed between multiple devices. In addition, any of the described units, modules, subsystems, or components may be implemented together or separately as discrete but interoperable logic devices of control circuitry. Depiction of different features as modules, subsystems, or units is intended to highlight different functional aspects and does not necessarily imply that such modules or units must be realized as hardware or software components and/or by a single device. Rather, specified functionality associated with one or more module, subsystem, or unit, as part of a control circuitry, may be performed by separate hardware or software components, or integrated within common or separate hardware or software components of control circuitry.

When implemented in software, the functionality ascribed to the systems, devices, and control circuitry described in this disclosure may be embodied as instructions on a physically embodied computer-readable medium such as RAM, ROM, NVRAM, EEPROM, FLASH memory, magnetic data storage media, optical data storage media, or the like, the medium being physically embodied in that it is not a carrier wave, as part of control circuitry. The instructions may be executed to support one or more aspects of the functionality described in this disclosure.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as falling within the scope of the claims, together with all equivalents thereof.

What is claimed is:

1. A system for measuring a force within a body, the system comprising:
a catheter comprising at least one LC circuit and at least one mass of high magnetic permeability material, each LC circuit of the at least one LC circuit comprising an inductor and a capacitor electrically in parallel, wherein the catheter is configured such that, responsive to the force, either of the at least one mass or each inductor of the at least one LC circuit is displaceable within the catheter relative to the other of the at least one mass or each inductor of the at least one LC circuit; and
control circuitry configured to:
measure, for each of the at least one LC circuit, a change in a resonance frequency of the LC circuit based on a change in distance between the inductor and the at least one mass, the change in distance responsive to the force; and
calculate at least one parameter of the force based on the change in the resonance frequency.

2. The system of claim 1, wherein the at least one parameter comprises a magnitude and a direction of the force.

3. The system of claim 2, further comprising a display, wherein the control circuitry is configured to graphically indicate on the display the magnitude and the direction of the force.

4. The system of claim 1, wherein the catheter further comprises a spring element located between each inductor of the at least one LC circuit and the at least one mass.

5. The system of claim 4, wherein the spring element is configured to:
permit the change in distance between each inductor of the at least one LC circuit and the at least one mass; and
resiliently reverse the change in distance upon removal of the force from the catheter.

6. The system of claim 4, wherein the control circuitry is configured to calculate the parameter of the force by using a function which relates the change in resonance frequency to the change in distance.

7. The system of claim 4, wherein the control circuitry is configured to calculate the parameter of the force by using a constant which relates the change in the change in distance to a value of the parameter of the force.

8. The system of claim 1, wherein the at least one LC circuit comprises three LC circuits, the three LC circuits circumferentially arrayed within the catheter.

9. The system of claim 1, wherein the high magnetic permeability material has a relative permeability greater than 1500.

10. The system of claim 1, wherein the at least one mass of high magnetic permeability material is passive and is not configured to be electrically energized in connection with measuring the force.

11. The system of claim 1, further comprising an additional mass of high magnetic permeability material, wherein the at least one mass is positioned either of proximal or distal with respect to the at least one LC circuit and the additional mass is positioned the other of proximal or distal with respect to the at least one LC circuit, and wherein the catheter is configured such that the additional mass is not displaceable within the catheter relative to each inductor of the at least one LC circuit.

12. The system of claim 1, wherein for each of the at least one LC circuit, the control circuitry is configured to deliver a continuous waveform, wherein the continuous waveform causes the LC circuit to oscillate; and the control circuitry is configured to measure the change in the resonance frequency by analyzing the oscillation in the LC circuit and determining whether the oscillation changes in frequency between pulses of the plurality of pulses.

13. The system of claim 1, further comprising a printed circuit board, wherein each of the at least one LC circuit is mounted on the printed circuit board.

14. The system of claim 13, wherein each inductor of the at least one LC circuit comprises a conductor formed into a flat radial spiral.

15. The system of claim 1, wherein, for each of the at least one LC circuit, the control circuitry is configured to deliver a plurality of pulses, wherein each pulse causes the LC circuit to oscillate, and the control circuitry is configured to measure the change in the resonance frequency by analyzing the oscillation in the LC circuit and determining whether the oscillation changes in frequency between pulses of the plurality of pulses.

16. The system of claim 1, wherein, for each LC circuit of the plurality of LC circuits, the inductance of the LC circuit changes based on the proximity of the at least one mass to the inductor of the circuit.

17. A system for measuring a force within a body, the system comprising:
a catheter comprising at least one sensor, at least one mass of high magnetic permeability material, and at least one spring element configured to permit movement within the catheter between the at least one sensor and the at least one mass responsive to the force; and
control circuitry configured to:
measure, for each sensor, a change in a resonance frequency of the sensor based on a change in distance between the sensor and the at least one mass, the change in distance responsive to the force; and
calculate at least one parameter of the force based on the change in the resonance frequency.

18. The system of claim 17, further comprising a display, wherein the at least one parameter comprises a magnitude and a direction of the force and the control circuitry is configured to graphically indicate on the display the magnitude and the direction of the force.

19. The system of claim 17, wherein the control circuitry is configured to calculate the at least one parameter of the force based at least in part on a spring constant for the spring element.

20. A system for measuring a force within a body, the system comprising:
a catheter comprising at least one sensor and an element located within the catheter, the element displaceable within the catheter relative to the at least one sensor; and
control circuitry configured to:
measure, for each of the at least one sensor, a change in a resonance frequency of the sensor based on a change in distance between the sensor and the element, the change in distance responsive to the force;
calculate at least one parameter of the force based on the change in the resonance frequency; and
output an indication of the at least one parameter of the force.
